(19) United States
(12) Patent Application Publication
(10) Pub. No.: US 2026/0043800 A1
(43) Pub. Date: Feb. 12, 2026

(54) FUNCTIONALIZED POROUS SUBSTRATES AND THEIR USE FOR DETECTING ANALYTES

(71) Applicant: Solventum Intellectual Properties Company, Maplewood, MN (US)

(72) Inventors: Deniz YUKSEL YURT, Saint Paul, MN (US); Semra COLAK ATAN, Saint Paul, MN (US); Nisha HOLLINGSWORTH, Saint Paul, MN (US); Ki-Joo SUNG, Saint Paul, MN (US); Katie F. WLASCHIN, Saint Paul, MN (US)

(73) Assignee: Solventum Intellectual Properties Company, Maplewood, MN (US)

(21) Appl. No.: 19/100,998

(22) PCT Filed: Jul. 21, 2023
(86) PCT No.: PCT/IB2023/057472

§ 371 Date: Feb. 4, 2025

Related U.S. Application Data
(60) Provisional application No. 63/398,079, filed on Aug. 15, 2022.

(51) Int. Cl. G01N 33/544; B01D 69/02; B01D 71/76; B01J 20/24; B01J 20/26; B01J 20/28

(52) U.S. Cl. CPC G01N 33/544; B01D 69/02; B01D 71/76; B01J 20/24; B01J 20/261; B01J 20/262; B01J 20/28023; B01J 20/28035; B01D 2325/0283

(57) ABSTRACT

Described herein is a multilayered article comprising a functionalized porous substrate. Such functionalized porous substrates can be used in the detection of analytes, wherein a reagent matrix comprising: (1) a plurality of first capture components, wherein the first capture component comprises a first analyte capture site and a porous substrate binding site; and (2) a plurality of a second capture component, wherein the second capture component comprises a second analyte capture site; wherein at least one of the first or second capture components comprises a detection medium; is contacted with an analyte and then disposed onto the functionalized porous substrate for analysis. In one embodiment, a novel monomer used to functionalize a substrate is described.

### TECHNICAL FIELD

Disclosed herein is a reaction matrix comprising at least two different capture components, which when contacted with an analyte of interest and a functionalized porous substrate result in detection.

### SUMMARY

Point of care (POC) devices are a powerful tool for rapid and inexpensive testing in clinical settings as well as the monitoring of food safety and environmental conditions. Most POC devices obviate the need for centralized laboratory testing and provide qualitative or semi-quantitative results without specialized equipment. There has been an increasing demand for POC testing worldwide due to persistent and emerging pandemics like tuberculosis, HIV/AIDS, and COVID-19.

One type of POC device is a lateral flow assay. In typical lateral flow assays, a liquid sample (or its extract) is added to the device and the capillary flow of a liquid is used to move the analyte of interest to other zones of the device for detection. Because capillary flow is used to move the analyte of interest to the detection zone, these assays can require wait times of 10-30 minutes to “read” the test result.

In vertical flow assays the sample is applied to the device and detection occurs at the point of application. Vertical flow assays are known to have a faster detection time than lateral flow assays, but are often more complicated for end-users, including multiple reaction steps (for example at least 2 different reaction reagents are used along with a tagging solution), which must be done in a certain order and numerous wash steps are needed to clear away reagents.

In one aspect, a kit for performing an assay for detection of an analyte in a sample is disclosed. The kit comprising:
(a) a porous substrate comprising a plurality of grafted groups to the porous substrate, wherein the plurality of grafted groups are derived from (i) an acidic monomer comprising at least one acidic group or salts thereof and a monovalent ethylenically unsaturated group selected from a (meth)acrylate or a (meth)acrylamide; (ii) a neutral hydrophilic monomer comprising at least one —O—CH₂CH₂— group and a monovalent ethylenically unsaturated group selected from a (meth)acrylate or a (meth)acrylamide; or (iii) combinations thereof; and
(b) a reagent matrix comprising:
   (1) a plurality of first capture components, wherein the first capture component comprises a first analyte capture site and a porous substrate binding site; and
   (2) a plurality of a second capture component, wherein the second capture component comprises a second analyte capture site;
   wherein at least one of the first or second capture components comprises a detection medium.

In another aspect, a method for detecting the presence or amount of an analyte in a sample is disclosed. The method comprising.
(a) in a vessel, combining the sample, a reagent matrix, and a carrier solution to form a test sample, the reagent matrix comprising:
   (1) a plurality of first capture component, wherein the first capture component comprises a first analyte capture site and a porous substrate binding site; and
   (2) a plurality of a second capture component, wherein the second capture component comprises a second analyte capture site; wherein at least one of the first or second capture components comprises a detection medium; and
   (3) optionally, a wetting or lysing agent:
(b) contacting the test sample to a porous substrate, wherein the porous substrate comprises a plurality of grafted groups to the porous substrate, wherein the plurality of grafted groups are derived from (i) an acidic monomer comprising at least one acidic group or salts thereof and a monovalent ethylenically unsaturated group selected from a (meth)acrylate or a (meth)acrylamide;
(ii) a neutral hydrophilic monomer comprising at least one —O—CH₂CH₂— group and a monovalent ethylenically unsaturated group selected from a (meth)acrylate or a (meth)acrylamide; or (iii) combinations thereof; and
(c) detecting a signal produced by aggregation of the analyte with the reagent matrix on the porous substrate.

In yet another aspect, a multilayered article is disclosed comprising:
(a) a porous substrate comprising a plurality of grafted groups to the porous substrate, wherein the plurality of grafted groups are derived from (i) an acidic monomer comprising at least one acidic group or salts thereof and a monovalent ethylenically unsaturated group selected from a (meth)acrylate or a (meth)acrylamide; (ii) a neutral hydrophilic monomer comprising at least one —O—CH₂CH₂— group and a monovalent ethylenically unsaturated group selected from a (meth)acrylate or a (meth)acrylamide; or (iii) combinations thereof; and
(b) an absorbent substrate thereon.

The above summary is not intended to describe each embodiment. The details of one or more embodiments of the invention are also set forth in the description below. Other features, objects, and advantages will be apparent from the description and from the claims.

### DETAILED DESCRIPTION

As used herein, the term
“a”, “an”, and “the” are used interchangeably and mean one or more; and “and/or” is used to indicate one or both stated cases may occur, for example A and/or B includes, (A and B) and (A or B);
“boronato” means a monovalent group of formula —B(OH)₂;
“carbonylimino” means a divalent group or moiety of formula —(CO)NR—, where R is hydrogen, alkyl (for example, selected from alkyl groups having from one to about four carbon atoms), or aryl (preferably, hydrogen);
“carboxy” means a monovalent group of formula —COOH;
“catenated atom” means an in-chain atom (rather than an atom of a chain substituent);
“catenated heteroatom” means an atom other than carbon (for example, oxygen, nitrogen, or sulfur) that replaces one or more carbon atoms in a carbon chain (for example, so as to form a carbon-heteroatom-carbon chain or a carbon-heteroatom-heteroatom-carbon chain);
“ethylenically unsaturated” means a group of formula —CY═CH₂ where Y is hydrogen, alkyl, cycloalkyl, or aryl;
“heteroatom” means an atom other than carbon or hydrogen;
“hydrogen bond acceptor” means a heteroatom selected from oxygen, nitrogen, and sulfur that has a lone electron pair;
“hydrogen bond donor” means a moiety consisting of a hydrogen atom covalently bonded to a heteroatom selected from oxygen, nitrogen, and sulfur;
“hydrogen bonding moiety” means a moiety comprising at least one hydrogen bond donor and at least one hydrogen bond acceptor;
“hydroxy” means a monovalent group of formula —OH;
“iminocarbonylimino” means a divalent group or moiety of formula —N(R)—C(O)—N(R)—, wherein each R is independently hydrogen, alkyl (for example, selected from alkyl groups having from one to about four carbon atoms), or aryl (preferably, at least one R is hydrogen; more preferably, both are hydrogen);
“iminothiocarbonylimino” means a divalent group or moiety of formula —N(R)—C(S)—N(R)—, wherein each R is independently hydrogen, alkyl (for example, selected from alkyl groups having from one to about four carbon atoms), or aryl (preferably, at least one R is hydrogen; more preferably, both are hydrogen);
“isocyanato” means a monovalent group of formula —N═C═O;
“oxycarbonylimino” means a divalent group or moiety of formula —O—C(O)—N(R)—, wherein R is hydrogen, alkyl (for example, selected from alkyl groups having from one to about four carbon atoms), or aryl (preferably, hydrogen);
“oxythiocarbonylimino” means a divalent group or moiety of formula —O—C(S)—N(R)—, wherein R is hydrogen, alkyl (for example, selected from alkyl groups having from one to about four carbon atoms), or aryl (preferably, hydrogen);
“phosphato” means a monovalent group of formula —OPO₃H₂;
“phosphono” means a monovalent group of formula —PO₃H₂;
“sulfato” means a monovalent group of formula —OSO₃H;
“sulfono” means a monovalent group of formula —SO₃H;
“thiocarbonylimino” means a divalent group or moiety of formula —(CS)NR—, where R is hydrogen, alkyl (for example, selected from alkyl groups having from one to about four carbon atoms), or aryl (preferably, hydrogen).

Also herein, recitation of ranges by endpoints includes all numbers subsumed within that range (e.g., 1 to 10 includes 1.4, 1.9, 2.33, 5.75, 9.98, etc.).

Also herein, recitation of “at least one” includes all numbers of one and greater (e.g., at least 2, at least 4, at least 6, at least 8, at least 10, at least 25, at least 50, at least 100, etc.).

As used herein, “comprises at least one of” A, B, and C refers to element A by itself, element B by itself, element C by itself, A and B, A and C, B and C, and a combination of all three.

In the present disclosure, a novel technique for detection of a particular analyte is disclosed.

In an aggregation-type assay, the target analyte binds to particular capture agents to form a detectable agglomerate. In the present disclosure, an aggregation-type assay is performed using a functionalized porous substrate in addition to capture components to improve the detection for a particular analyte. The method of the present disclosure is a simplified process for a vertical flow type assay, wherein a single step can be used to lyse the sample, and capture and label the target analyte. The method is simple, fast, and can provide detection limits comparable or better than commercially available tests.

In the method of the present application, the target analyte is used to complex two different capture components, wherein at least one of the capture components can also bind to a functionalized substrate, thereby capturing the agglomerate onto the substrate surface.

### Reagent Matrix

The reagent matrix of the present disclosure comprises at least two different components that can capture the target analyte. The analyte (or target analyte) is the compound or composition of interest to be detected. The analyte can be biologically-derived or present in a biologically-derived testing sample fluid. Examples of such analytes may include therapeutic drugs, drugs of abuse, pharmaceutical metabolites, hormones, peptides, polypeptides, proteins including immunoglobulins, polysaccharides, nucleic acids, and combinations thereof. In another embodiment, the analyte may be an agent of environmental interest, such as a pest control product, an environmental toxin, a halogenated substance, or dioxins and furans. In another embodiment, the analyte may be an agent of food safety interest, such as pathogens (e.g., bacteria, virus, fungi, etc.), allergens, pesticides, genetically modified organisms, and toxins.

The first capture component comprises a first site for capture of the analyte and a second capture site for the binding of the functionalized porous substrate. The first capture site is capable of recognizing a particular spatial and/or chemical structure of the analyte. The second site is capable of recognizing a particular spatial and/or chemical structure of the functionalized porous substrate layer.

The second capture component comprises a capture site which also is capable of recognizing a particular spatial and/or chemical structure of the analyte.

In one embodiment, the capture site may be an antibody. As used herein, the term antibody covers not only antibodies, but any polypeptide or protein having a binding domain which is, or is homologous to, an antibody binding domain. These can be derived from natural sources, or they may be partly or wholly synthetically produced. Examples of antibodies are the immunoglobulin isotypes and their isotypic subclasses; fragments which comprise an antigen binding domain such as Fab, scFv, Fv, dAb, Fd; and diabodies. Antibodies useful in the present disclosure include those specifically reactive with the target analyte. Antibody capture sites are preferable when testing of biological samples. Such antibodies are preferably IgG or IgM antibodies or mixtures thereof, which are essentially free of association with antibodies capable of binding with non-analyte molecules. The antibodies may be polyclonal or monoclonal and are commercially available or may be obtained by mouse ascites, tissue culture or other techniques known to the art. The use of mixtures of monoclonal antibodies of differing antigenic specificities or of monoclonal antibodies and polyclonal antibodies may be desired. A typical description of hybridoma procedure for the production of monoclonal antibodies may be found for example in Wands, et al., Gastroenterology 80 pages 225-232 (1981); and U.S. Pat. No. 4,515,893 issued to Kung, et al. It is further contemplated that fragments of antibody molecules may be used as specific binding reagents according to the present disclosure including half antibody molecules and Fab, Fab′ or F(ab′)2 fragments known in the art.

In another embodiment, the capture site is a capture protein such as an engineered protein, peptide aptamers, or affimer proteins. A plethora of protein engineering methods are known in the art to generate peptides and proteins with enhanced or novel functions. One such methodology is directed evolution, which involves creation of a random library displaying a broad set of protein variants. The library is then displayed using one of the various technologies available e.g., phage, ribosome, mRNA, or cell surface display. Following selections, affinity maturation is obtained by adding diversity back to the library by DNA modification (e.g., error prone PCR, DNA shuffling, etc.). The cyclic selection process is repeated until the desired binders are produced. In addition to molecular biology-based approaches, chemical combinatorial methods such as DNA-encoded libraries and one-bead-one-compound libraries can be used to create high affinity binding peptides. Other approaches to obtain new binding domains also include semirational design and computational techniques. An overview of the techniques mentioned here can be found in Banta et al. in Annu Rev. Biomed Eng., vol. 15, pages 93-113 (2013); and Zhao et al. in Expert Opin. Drug Discov., vol. 14, pages 735-753 (2019).

In another embodiment, the capture site is an aptamer, such as DNA, RNA, or peptide aptamers. RNA and DNA aptamers bind to their targets with high selectivity and sensitivity due to their structural confirmation. The aptamers can be produced by techniques known in the art such as SELEX (systematic evolution of ligands by exponential enrichment). See Ellington et al. in Nature, v. 346, pages 818-822 (1990), and Tuerk, et al. in Science, v. 249, pages 505-510 (1990). Peptide aptamers is another alternative binding molecule where a 5-20 residue peptide is typically grafted onto a neutral scaffold which undergoes a selection procedure. Peptide aptamers can be produced and selected using display strategies known in the art. See Reverdatto et al. in Curr. Top. Med. Chem. v. 15, iss. 12, pages 1082-1101 (2015).

The first capture component, the second capture component, or both comprise a detection medium. The detection medium may be any molecule or particle bound or conjugated to the capture site, which can enable detection. The signal may be one that is detected visually (for example by eye) or one that is detected with an instrument. The detection medium may be a colorant, a photoluminescent substance, a chemiluminescent substance, a radio-label, a magnetic material, or combinations thereof.

The first and second capture sites are selected to have a specific binding affinity for different portions of the analyte, thereby sandwiching the analyte therebetween. The capture sites may be naturally derived or synthetically produced. The first capture site specifically binds to and is therefore complementary to a particular spatial and/or chemical structure of the analyte, while the second capture site specifically binds to and is therefore complementary to a particular spatial and/or chemical structure of another portion the analyte. It is known in the art, how to select such sandwich pairs. See for example, a review article by Mirica et al. in Front. Bioeng. Biotechnol., vol 10, article 922772, June 2022.

In one embodiment, the capture component comprises a particle, wherein the capture site is bound or conjugated on or to the particle via passive adsorption or covalent attachment as known in the art. Typically, these particles may comprise a synthetic polymer (such as a latex), glass, metal, metal oxide, liposomes, pollen spores, red blood cells, carbohydrates (such as dextans, agarose, or cellulose), microganisms including viruses, and combinations thereof. In one embodiment, the particles comprise nanocellulose or latex. Latexes are commercially available and the polymer particles therein may be derived from acrolein, acrylate, methyl acrylate, methacrylate, methyl methacrylate, glycidyl methacrylate, styrene, vinyl toluene, and t-butyl styrene monomers and mixtures thereof. In some embodiments, the polymer particles of the latex may optionally containing crosslinking agents such as divinyl benzene and butadiene. Techniques for preparing such latexes are well-known as are surface modifications used to attach binding pair members to the particle surfaces. Exemplary U.S. patents that describe either latex particles, capture sites that can be attached to the particles, and/or coupling methods for attaching the capture sites to the particle surfaces include U.S. Pat. No. 4,064,088 (Renner); U.S. Pat. No. 4,210,723 (Dorman, et al.); U.S. Pat. No. 4,264,766 (Fisher); U.S. Pat. No. 3,857,931 (Hager); U.S. Pat. No. 4,253,844 (Limet et al.); and U.S. Pat. No. 4,397,960 (Moussebois, et al.), each herein incorporated by reference.

In one embodiment, a dye is added to the particle, such that when a substantial amount of particles agglomerate at the surface of the porous substrate, a signal can be visually observed.

The size of the particles and the pores/porosity of the porous substrate are selected such that unbound particles can pass through the porous substrate, while when the analyte of interest is sandwiched between the first and second capture components and the sandwiched analyte does not pass through the porous substrate. In one embodiment, the particles have an average diameter of at least 0.1, 0.5, or even 1 micrometer and at most 100, 75, 50, 25, 15, or even 10 micrometers. The particle size may be determined using techniques known in the art such as scanning electron microscope or light scattering detection.

Advantageously, the reagent mixture can be substantially free liquid (in other words comprising less than 5, 3, 2, 1, 0.5, or even 0.1% by weight of a liquid, such as water, alcohol, etc. or even no liquid is detectable). The reagent mixture may be lyophilized, or similarly dried. The reagent mixture being substantially free of water can enable improved shelf-life.

In one embodiment, the reagent matrix further comprises a wetting or lysing agent, such as a solvent (e.g., alcohol) or a surfactant.

### Porous Substrate

In one embodiment, a test sample is applied to a porous substrate. Although not wanting to be bound by theory, it is believed that capture components interact with the target analyte and upon application to the porous substrate, the aggregated product is concentrated at the surface of the porous substrate while the unbound reagents are drawn away from the surface.

The porous substrate of the present disclosure is a layer comprising a series of interconnected pores from a first major surface of the porous substrate to an opposing second major surface of the porous substrate.

The porous substrate is an organic material, preferably a polymeric material, which can be a porous film or nonwoven.

In one embodiment, the porous substrate is a nonwoven web, which may include nonwoven webs manufactured by any of the commonly known processes for producing nonwoven webs. As used herein, the term “nonwoven web” refers to a fabric that has a structure of individual fibers or filaments which are randomly and/or unidirectionally interlaid in a mat-like fashion. For example, the fibrous nonwoven web can be made by carded, air laid, spunlaced, spunbonding or melt-blowing techniques or combinations thereof. Spunbonded fibers are typically small-diameter fibers that are formed by extruding molten thermoplastic polymer as filaments from a plurality of fine, usually circular capillaries of a spinneret with the diameter of the extruded fibers being rapidly reduced. Meltblown fibers are typically formed by extruding the molten thermoplastic material through a plurality of fine, usually circular, die capillaries as molten threads or filaments into a high velocity, usually heated gas (e.g., air) stream, which attenuates the filaments of molten thermoplastic material to reduce their diameter. Thereafter, the meltblown fibers are carried by the high velocity gas stream and are deposited on a collecting surface to form a web of randomly disbursed meltblown fibers. Any of the non-woven webs may be made from a single type of fiber or two or more fibers that differ in the type of thermoplastic polymer, thickness thereof, or both. Further details of manufacturing methods of useful nonwoven webs have been described by Wente in Indus. Eng. Chem., v. 48, pages 1342-1346 (1956). In one embodiment, the microfibers have an effective fiber diameter of at least 0.5 and at most 16 micrometers.

In some embodiments, the porous substrate is a porous membrane such as a thermally-induced phase separation (TIPS) membrane. TIPS membranes are often prepared by forming a homogenous solution of a thermoplastic material and a diluent, and optionally including a nucleating agent, by mixing at elevated temperatures in plastic compounding equipment, e.g., an extruder. The solution can be shaped by passing through an orifice plate or extrusion die, and upon cooling, the thermoplastic material crystallizes and phase separates from the diluent. The crystallized thermoplastic material is often stretched. The diluent is optionally removed either before or after stretching, leaving a porous polymeric structure. Porous membranes are further disclosed in U.S. Pat. No. 4,539,256 (Shipman), U.S. Pat. No. 4,726,989 (Mrozinski), U.S. Pat. No. 4,867,881 (Kinzer), U.S. Pat. No. 5,120,594 (Mrozinski), U.S. Pat. No. 5,260,360 (Mrozinski et al.), U.S. Pat. No. 5,962,544 (Waller), and U.S. Pat. No. 6,096,293 (Mrozinski et al.) all of which are assigned to 3M Company (St. Paul, MN), and each incorporated herein by reference.

In some embodiments, the substrate is a porous membrane such as a solvent-induced phase separation (SIPS) membrane. SIPS membranes are often made by preparing a homogeneous solution of a polymer in first solvent(s), casting the solution into desired shape, e.g., flat sheet or hollow fiber, contacting the cast solution with another second solvent that is a non-solvent for the polymer, but a solvent for the first solvent (i.e., the first solvent is miscible with the second solvent, but the polymer is not). Phase separation is induced by diffusion of the second solvent into the cast polymer solution and diffusion of the first solvent out of the polymer solution and into the second solvent, thus precipitating the polymer. The polymer-lean phase is removed and the polymer is dried to yield the porous structure. SIPS is also called Phase Inversion, or Diffusion-induced Phase Separation, or Nonsolvent-induced Phase Separation, such techniques are commonly known in the art.

Useful porous substrates include symmetric, asymmetric, or multizone membranes, as well as multiple layers of such membranes. A symmetric membrane is one having substantially the same average pore size and/or porosity throughout its thickness. An asymmetric membrane is a membrane having a linear or non-linear gradient in average pore size and/or porosity extending from one major surface to an opposing major surface of the fluoroplastic substrate. In other words, the ratio of the average pore size of the one major surface with the larger pores to the average pore size of the opposing surface is greater than 3 or even greater than 4. A multizone membrane is a membrane having two or more substantially distinct through-thickness zones, or layers having different average pore sizes and/or different porosities. Multizone membranes are often designated by the number of layers or zones, (e.g., a 2-zone membrane has two substantially distinct zones having different average pore sizes or different porosities).

The porous substrate is an organic material, preferably a polymeric material. In one embodiment, the porous substrate may be formed from any suitable polymeric material. Suitable polymeric materials include polyolefins, poly(isoprenes), poly(butadienes), fluorinated polymers, chlorinated polymers, polyamides, polyimides, polyethers, poly(ether sulfones), poly(sulfones), poly(vinyl acetates), polyesters such as poly(lactic acid), copolymers of vinyl acetate such as poly(ethylene)-co-poly(vinyl alcohol), poly(phosphazenes), poly(vinyl esters), poly(vinyl ethers), poly(vinyl alcohols), poly(carbonates), fiberglass, cellulose, and the like, and combinations thereof.

Suitable polyolefins include poly(ethylene), poly(propylene), poly(1-butene), copolymers of ethylene and propylene, alpha olefin copolymers (such as copolymers of ethylene or propylene with 1-butene, 1-hexene, 1-octene, and 1-decene), poly(ethylene-co-1-butene), poly(ethylene-co-1-butene-co-1-hexene), and the like, and combinations thereof.

Suitable fluorinated polymers include poly(vinyl fluoride), poly(vinylidene fluoride), copolymers of vinylidene fluoride (such as poly(vinylidene fluoride-co-hexafluoropropylene)), copolymers of chlorotrifluoroethylene (such as poly(ethylene-co-chlorotrifluoroethylene)), and the like, and combinations thereof.

Suitable polyamides include poly(iminoadipolyliminohexamethylene), poly(iminoadipolyliminodecamethylene), polycaprolactam, and the like, and combinations thereof. Suitable polyimides include poly(pyromellitimide), and the like, and combinations thereof.

Suitable poly(ether sulfones) include poly(diphenylether sulfone), poly(diphenylsulfone-co-diphenylene oxide sulfone), and the like, and combinations thereof.

Suitable copolymers of vinyl acetate include poly(ethylene-co-vinyl acetate), such copolymers in which at least some of the acetate groups have been hydrolyzed to afford various poly(vinyl alcohols), and the like, and combinations thereof.

In one exemplary embodiment, the porous substrate has an average pore size that is greater than 200, 500, 750, 1000, 2000, 3000, or even 5000 nanometers (nm). In one exemplary embodiment, the porous substrate has an average pore size that is less than about 100, 50, 25, 20, 10, 5, 3, or even 2 μm (micrometers). The average pore size of the porous substrate can be optimized for the particular capture components used and the target analyte. The average pore size can be measured using techniques known in the art, for example, optical microscopy, computed tomography (CT) scanning, or liquid porosimetry.

The porous substrates of the present disclosure comprise a surface functionalization, which interacts with at least one of the capture components. The surface functionalization is a plurality of grafted groups extending from the surface of the porous substrate. The grafted groups are derived from (i) an acidic monomer comprising at least one acidic group or salts thereof and a monovalent ethylenically unsaturated group selected from a (meth)acrylate or a (meth)acrylamide; and/or (ii) a neutral hydrophilic monomer comprising at least one —O—CH₂CH₂— group and a monovalent ethylenically unsaturated group selected from a (meth)acrylate or a (meth)acrylamide.

Monomers suitable for use in preparing the functionalized porous substrate include those that consist of (a) at least one monovalent ethylenically unsaturated group selected from a (meth)acrylate or a (meth)acrylamide; (b) at least one monovalent ligand functional group selected from acidic groups, salts of acidic group, and neutral hydrophilic groups comprising at least one —O—CH₂CH₂— group; and (c) optionally, a multivalent spacer group that is directly bonded to the monovalent groups so as to link at least one ethylenically unsaturated group and at least one ligand functional group by a chain of at least six catenated atoms. The monomers can be in a neutral state but can also be negatively (if acidic) charged under some pH conditions.

The monovalent ethylenically unsaturated group of the monomer(s) can be represented by the formula CH₂═CYC(═O)O— or CH₂═CYC(═O)NH₂— wherein Y is hydrogen, or alkyl, cycloalkyl, or aryl. Preferred ethylenically unsaturated groups include ethenyl, 1-alkylethenyl, and combinations thereof (that is, Y is preferably hydrogen or alkyl; more preferably, Y is hydrogen or C₁ to C₄ alkyl; most preferably, Y is hydrogen or methyl). The monomer(s) can comprise a single ethylenically unsaturated group or multiple ethylenically unsaturated groups (for example, two or three or up to as many as 6), which can be the same or different in nature (preferably, the same). The monomer(s) preferably have only one ethylenically unsaturated group.

The monovalent ligand functional group of the monomer(s) can be selected from acidic groups, salts of acidic group, and neutral hydrophilic groups. Suitable acidic ligand functional groups include those that exhibit at least a degree of acidity (which can range from relatively weak to relatively strong), as well as salts thereof. Such ligand functional groups include those commonly utilized as ion exchange or metal chelate type ligands. Suitable neutral ligand functional groups include those that comprising at least one —O—CH₂CH₂— group.

Useful ligand functional groups include heterohydrocarbyl groups and other heteroatom-containing groups. For example, useful acidic ligand functional groups can comprise one or more heteroatoms selected from oxygen, nitrogen, sulfur, phosphorus, boron, and the like, and combinations thereof. Useful salts of acidic groups include those having counter ions selected from alkali metal (for example, sodium or potassium), alkaline earth metal (for example, magnesium or calcium), ammonium, and tetraalkylammonium ions, and the like, and combinations thereof.

The monomer(s) can comprise a single ligand functional group or multiple ligand functional groups (for example, two or three or up to as many as 6), which can be the same or different in nature (preferably, the same). The ligand functional group(s) are preferably selected from carboxy, phosphono, phosphato, sulfono, sulfato, boronato, and combinations thereof. More preferred ligand functional group(s) include carboxy, phosphono, sulfono, and combinations thereof.

If a spacer group is used, the multivalent spacer group of the monomer(s) can be directly bonded to the monovalent groups so as to link at least one ethylenically unsaturated group and at least one ligand functional group by a chain of at least six catenated atoms. Thus, the chain can comprise 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or more catenated atoms (for example, including up to as many as 40 or 50). The chain preferably comprises at least seven catenated atoms (more preferably, at least eight; most preferably, at least nine, ten, eleven, or twelve) and/or comprises no more than about 30 catenated atoms (more preferably, no more than about 25; even more preferably, no more than about 20; most preferably, no more than about 16).

Preferred multivalent spacer groups comprise at least one hydrogen bonding moiety, which is defined above as a moiety comprising at least one hydrogen bond donor and at least one hydrogen bond acceptor (both of which are heteroatom-containing, as described above). Thus, preferred multivalent spacer groups include heteroatom-containing hydrocarbon groups (more preferably, catenated heteroatom-containing hydrocarbon groups). More preferred spacer groups comprise at least two hydrogen bonding moieties or comprise at least one hydrogen bonding moiety and at least one hydrogen bond acceptor that is distinct from (not part of) the hydrogen bonding moiety.

Preferred hydrogen bonding moieties include those that comprise at least two hydrogen bond donors (for example, donors such as imino, thio, or hydroxy), at least two hydrogen bond acceptors (for example, acceptors in the form of carbonyl, carbonyloxy, or ether oxygen), or both. For example, an iminocarbonylimino moiety (having two N—H donors and at least two acceptors in the form of two lone electron pairs on carbonyl) can sometimes be preferred over a single iminocarbonyl moiety. Preferred spacer groups include those that comprise at least one iminocarbonylimino moiety (more preferably, in combination with at least one acceptor such as carbonyloxy), at least two iminocarbonyl moieties, or a combination thereof.

The hydrogen bond donor and hydrogen bond acceptor of the hydrogen bonding moiety can be adjacent (directly bonded) to each other or can be non-adjacent (preferably, adjacent or separated by a chain of no more than about 4 catenated atoms; more preferably, adjacent). The heteroatoms of the hydrogen bond donor and/or hydrogen bond acceptor can be located in the chain of catenated atoms of the spacer group or, alternatively, can be located in chain substituents.

Although hydrogen bond donors can also function as hydrogen bond acceptors (through a lone electron pair of the donor's heteroatom), the hydrogen bonding moiety preferably comprises distinct donor and acceptor moieties. This can facilitate intramolecular (intermonomer) hydrogen bond formation. Although not wishing to be bound by theory, such intramolecular hydrogen bonds between adjacent monomer repeat units in the polymer molecule may contribute to at least a degree of multivalent spacer group stiffening, which may facilitate presentation of the ligand functional group(s) for interaction with a target biomaterial.

Preferred hydrogen bonding moieties include carbonylimino, thiocarbonylimino, iminocarbonylimino, iminothiocarbonylimino, oxycarbonylimino, oxythiocarbonylimino, and the like, and combinations thereof. More preferred hydrogen bonding moieties include carbonylimino, iminocarbonylimino, oxycarbonylimino, and combinations thereof (most preferably, carbonylimino, iminocarbonylimino, and combinations thereof). Preferred multivalent spacer groups include those that are divalent, trivalent, or tetravalent (more preferably, divalent or trivalent; most preferably, divalent).

A class of useful acidic monomers includes those represented by the following general formula

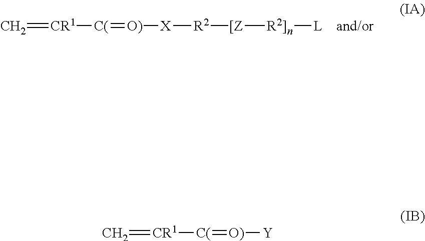

wherein
   R¹ is selected from hydrogen, alkyl, cycloalkyl, aryl, and combinations thereof;
   each R² is independently selected from hydrocarbylene, heterohydrocarbylene, and combinations thereof;
   X is —O— or —NR³—, where R³ is selected from hydrogen, hydrocarbyl, heterohydrocarbyl, and combinations thereof;
   Y is —OH;
   Z is heterohydrocarbylene comprising at least one hydrogen bond donor, at least one hydrogen bond acceptor, or a combination thereof;
   n is an integer of 0 or 1; and
   L is a heteroatom-containing group comprising at least one monovalent ligand functional group selected from acidic groups, salt of acidic group.

Preferably, R¹ is hydrogen or alkyl (more preferably, hydrogen or C₁ to C₄ alkyl; most preferably, hydrogen or methyl); each R² is independently hydrocarbylene (more preferably, independently alkylene); X is —O— or —NR³—, where R³ is hydrogen; Z is heterohydrocarbylene comprising at least one moiety selected from carbonyl, carbonylimino, carbonyloxy, ether oxygen, thiocarbonylimino, iminocarbonylimino, iminothiocarbonylimino, oxycarbonylimino, oxythiocarbonylimino, and combinations thereof (more preferably, selected from carbonyl, carbonylimino, carbonyloxy, ether oxygen, iminocarbonylimino, oxycarbonylimino, and combinations thereof; even more preferably, selected from carbonylimino, carbonyloxy, ether oxygen, iminocarbonylimino, and combinations thereof; most preferably, selected from carbonylimino, iminocarbonylimino, and combinations thereof); n is an integer of 1; and/or L is a heteroatom-containing group comprising at least one ligand functional group selected from carboxy, phosphono, phosphato, sulfono, sulfato, boronato, and combinations thereof (more preferably, selected from carboxy, phosphono, sulfono, and combinations thereof).

Such monomers can be prepared by known synthetic methods or by analogy to known synthetic methods. For example, amino group-containing carboxylic, sulfonic, or phosphonic acids can be reacted with ethylenically unsaturated compounds that comprise at least one group that is reactive with an amino group. Similarly, ligand functional group-containing compounds that also contain a hydroxy group can be reacted with ethylenically unsaturated compounds that comprise at least one group that is reactive with a hydroxy group, optionally in the presence of a catalyst. Preferred monomers are (meth)acryloyl-functional. (As used herein, the term “(meth)acryloyl-functional” refers to acryloyl-functional and/or methacryloyl-functional; similarly, the term “(meth)acrylate” refers to an acrylate and/or a methacrylate).

Representative examples of useful monomers include those derived from the reaction of an alkenyl azlactone of general Formula II

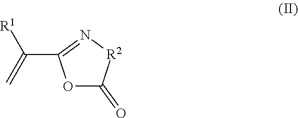

or an ethylenically unsaturated isocyanate of general Formula III

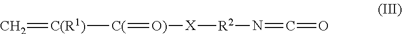

with a ligand functional group-containing compound of general Formula IV

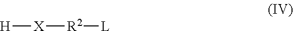

to produce monomer of general Formula I (wherein R¹, X, R², and L in Formulas II, III, and/or IV are as defined above for Formula I). Representative examples of useful alkenyl azlactones of Formula II include 4,4-dimethyl-2-vinyl-4H-oxazol-5-one (vinyldimethylazlactone, VDM), 2-isopropenyl-4H-oxazol-5-one, 4,4-dimethyl-2-isopropenyl-4H-oxazol-5-one, 2-vinyl-4,5-dihydro-[1,3]oxazin-6-one, 4,4-dimethyl-2-vinyl-4,5-dihydro-[1,3]oxazin-6-one, 4,5-dimethyl-2-vinyl-4,5-dihydro-[1,3]oxazin-6-one, and the like, and combinations thereof. Representative examples of ethylenically unsaturated isocyanates of general Formula III include 2-isocyanatoethyl (meth)acrylate (IEM or IEA), 3-isocyanatopropyl (meth)acrylate, 4-isocyanatocyclohexyl (meth)acrylate, and the like, and combinations thereof.

Representative examples of useful ligand functional group-containing compounds of general Formula IV include amino group-containing carboxylic, sulfonic, boronic, and phosphonic acids and combinations thereof. Useful amino carboxylic acids include α-amino acids (L-, D-, or DL-α-amino acids) such as glycine, alanine, valine, proline, serine, phenylalanine, histidine, tryptophan, asparagine, glutamine, N-benzylglycine, N-phenylglycine, sarcosine, and the like; β-aminoacids such as β-alanine, β-homoleucine, β-homoglutamine, β-homophenylalanine, and the like; other α,ω-aminoacids such as γ-aminobutyric acid, 6-aminohexanoic acid, 11-aminoundecanoic acid, peptides (such as diglycine, triglycine, tetraglycine, as well as other peptides containing a mixture of different aminoacids), and the like; and combinations thereof. Useful amino sulfonic acids include aminomethanesulfonic acid, 2-aminoethanesulfonic acid (taurine), 3-amino-1-propanesulfonic acid, 6-amino-1-hexanesulfonic acid, and the like, and combinations thereof. Useful aminoboronic acids include m-aminophenylboronic acid, p-aminophenylboronic acid, and the like, and combinations thereof. Useful aminophosphonic acids include 1-aminoethylphosphonic acid, 2-aminoethylphosphonic acid, 3-aminopropylphosphonic acid, and the like, and combinations thereof. Useful compounds of Formula IV containing more than one ligand functional group include aspartic acid, glutamic acid, α-aminoadipic acid, iminodiacetic acid, N_{α},N_{α}-bis(carboxymethyl)lysine, cysteic acid, N-phosphonomethylglycine, and the like, and combinations thereof.

Representative examples of other useful ligand functional group-containing compounds of general Formula IV include compounds comprising a hydroxy group and an acidic group. Specific examples include glycolic acid, lactic acid, 6-hydroxyhexanoic acid, citric acid, 2-hydroxyethylsulfonic acid, 2-hydroxyethylphosphonic acid, and the like, and combinations thereof.

Many of the above-described ligand functional group-containing compounds of general Formula IV are commercially available. Still other useful ligand functional group-containing compounds of general Formula IV can be prepared by common synthetic procedures. For example, various diamines or aminoalcohols can be reacted with one equivalent of a cyclic anhydride to produce an intermediate ligand functional group-containing compound comprising a carboxyl group and an amino or hydroxy group.

Useful monomers can also be prepared by the reaction of ligand functional group-containing compounds of general Formula IV with ethylenically unsaturated acyl halides (for example, (meth)acryloyl chloride). In addition, useful monomers can be prepared by reaction of hydroxy- or amine-containing (meth)acrylate or (meth)acrylamide monomers with a cyclic anhydride to produce carboxyl group-containing monomers.

Preferred monomers include monomers prepared from the reaction of alkenyl azlactones with aminocarboxylic acids, monomers prepared from the reaction of alkenyl azlactones with aminosulfonic acids, monomers prepared from the reaction of ethylenically unsaturated isocyanates with aminocarboxylic acids, monomers prepared from the reaction of ethylenically unsaturated isocyanates with aminosulfonic acids, and combinations thereof.

In one embodiment, exemplary acid type monomers include:

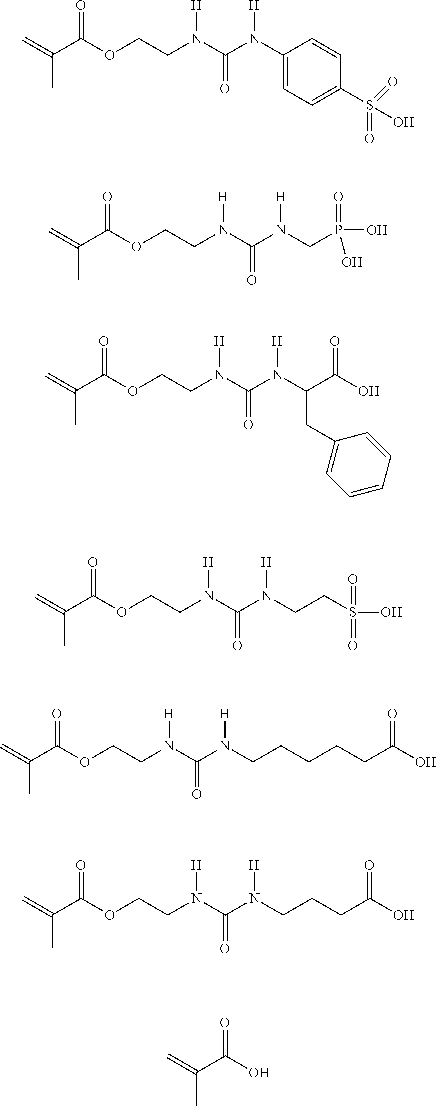

and salts thereof.

A class of useful monomers includes those represented by the following general formula

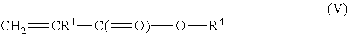

wherein
   R¹ is selected from hydrogen, alkyl, cycloalkyl, aryl, and combinations thereof; and
   R⁴ is H, or an oxy-hydrocarbyl,
wherein the monomer according to Formula (V) comprises no more than six —O—CH₂CH₂— groups.

In one embodiment, R⁴ is H, —(CH₂CH₂O)ᵣH wherein r is 1, 2, 3, 4, 5, or 6; —(CH₂CH₂O)ᵣR⁵ wherein r is 1, 2, 3, 4, 5, or 6 and R⁵ is methyl, ethyl or propyl. The monomer according to Formula (V) are readily commercially available or can be synthesized as described above using common synthetic procedures.

In one embodiment, exemplary hydrophilic neutral-type monomers include:

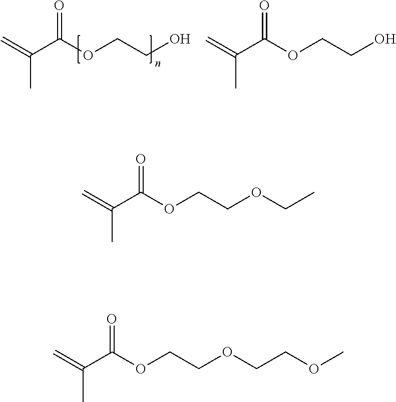

Wherein n is 5 or 6.

Optionally, the monomer(s) can be copolymerized with one or more (meth)acryloyl comonomer(s) containing at least two free radically polymerizable groups. Such multifunctional (meth)acryloyl comonomer(s) (including multifunctional (meth)acrylate(s) and (meth)acrylamide(s)) can be incorporated in a blend of polymerizable monomer(s) generally in only relatively small amounts (for example, from about 0.1 to about 5 percent by weight, based upon the total weight of monomer(s) and comonomer(s)) to impart a degree of branching and/or relatively light crosslinking to a resulting copolymer. Higher amounts can be used for certain applications, but it should be understood that the use of higher amounts may reduce binding capacity for a target analyte.

Useful multifunctional (meth)acryloyl comonomers include di(meth)acrylates, tri(meth)acrylates, tetra(meth)acrylates, multifunctional (meth)acrylamides, and the like, and combinations thereof. Such multifunctional (meth)acryloyl comonomers include ethyleneglycol di(meth)acrylate, 1,6-hexanediol di(meth)acrylate, poly(ethylene glycol) di(meth)acrylates, polybutadiene di(meth)acrylate, polyurethane di(meth)acrylates, propoxylated glycerin tri(meth)acrylate, methylenebisacrylamide, ethylenebisacrylamide, hexamethylenebisacrylamide, diacryloylpiperazine, and the like, and combinations thereof.

For example, the monomer(s) optionally can be copolymerized with one or more hydrophilic comonomer(s) comprising at least one alkenyl group (preferably, a (meth)acryloyl group) and a hydrophilic group (including poly(oxyalkylene) groups) in order to impart a degree of hydrophilicity to the porous substrate. Suitable hydrophilic comonomers include acrylamide, dimethylacrylamide, hydroxyethyl(meth)acrylate, hydroxypropyl(meth)acrylate, glycidyl methacrylate, polyethyleneglycolmono(meth)acrylate, 2-hydroxyethylacrylamide, N-vinylpyrrolidone, and the like, and combinations thereof. Such monomers may be used to enhance the grafting performance of the acidic monomers.

In one embodiment, the monomers are bound to the porous substrate. In one embodiment, the monomers of interest may be polymerized in the presence of the porous substrate, so as to graft them onto the porous substrate. Such a process is known in the art, as described in U.S. Pat. No. 9,958,364 (Rasmussen et al.). In one embodiment, the acidic monomer and/or neutral hydrophilic monomers of the present disclosure are subjected to, for example, to radiation (e.g., electron beam radiation) in the presence of a porous substrate. A first monomer is attached to the surface of the porous substrate creating a radical that reacts with a second monomer to form another radical that reacts with a third monomer, etc. In this way, a polymer can be grafted to the surface of the porous substrate. In an alternative, although not necessarily preferred, method for the grafting of the monomers onto the porous substrate, the porous substrate can be grafted, in a first step, with a monomer of general Formula II or general Formula III, or a mixture thereof. The grafted substrate can then, in a second step, be reacted with a first acidic group-containing compound of general Formula IV or mixture of two or more such compounds. Alternatively, in this second step, a portion of the grafted product of the first step can be reacted with a first acidic group-containing compound and the remaining portion of the grafted product can be reacted with a second acidic group-containing compound. The first step can be carried out using any of the common processes known for grafting of monomers to substrates (e.g., by using UV, gamma, and/or e-beam irradiation), with the precaution that solvents that are not reactive with the monomers of Formulas II and III are used. In the second step, a solvent that does not react with the azlactone group or isocyanate group of the grafted polymer, but that does dissolve the acidic group-containing compound of Formula IV is chosen. Such precautions are taken to minimize competing hydrolysis or solvolysis of the very reactive azlactone and isocyanate groups. Optionally, the acidic group-containing compound may be neutralized (converted to the salt form) prior to or subsequent to reaction.

Following polymerization, washing, and drying of the graft substrate, typical total weight gains by the porous substrate generally can be in the range of about 5 percent (%) to about 30% (preferably, in the range of about 10% to about 25%; more preferably, in the range of about 12% to about 20%). Polymerization of the monomer(s) in the presence of a porous substrate can produce a polymer-bearing porous substrate. The polymer can be in the form of a coating or, in preferred embodiments, the polymer can be grafted (covalently bonded) to the surface of the porous substrate. (If desired, the polymerization can be carried out separately and the resulting polymer then coated (optionally in the presence of suitable crosslinker) or grafted or otherwise applied to the porous substrate, but this is generally less preferred.)

In some biomaterial assays, proteins are immobilized onto the surface of substrates to detect analytes. In the present application, no immobilized proteins are bound to the surface of the substrate prior to contact with the reagent matrix. In one embodiment, the porous substrate is substantially free (i.e., comprises less than 0.5% by weight, or even no detectable amount) of immobilized proteins prior to contact with the reagent matrix.

In one embodiment, a plurality of cellulose nanofibrils is contacted with the test sample, which may enhance the detection of the analyte. The plurality of cellulose nanofibrils have be present in the reagent matrix or maybe added to the test sample before contact with the porous substrate layer. Cellulose nanofibrils are a particular type of cellulose particle. As used herein cellulose nanofibrils includes fibrillated cellulose (both nanofibrillated cellulose and microfibrillated cellulose).

Cellulose nanofibrils can be produced from cellulosic materials, such as wood pulp, bacteria, cellulose-containing sea animals (e.g., tunicate), or cotton, with wood pulp, the most commonly used.

The cellulose nanofibrils can be made using mechanical treatments, such as high-pressure homogenization, high-energy ball mills, microfluidizers, ultra-low crushing, and other such method; enzymatic treatments; and/or chemical treatments such as strong acid hydrolysis, oxidation, chemical functionalization, or combinations thereof.

Cellulose nanofibrils are constituted of cellulose, a linear polymer of beta (1 to 4) linked D-glucose units, the chains of which arrange themselves to form crystalline and amorphous domains.

The physical dimensions of cellulose nanofibrils can vary depending on the raw material and how it was treated. Typically, the microfibrillated cellulose comprises long thin fibers with a large size distribution, including individual fibers with a nanometer diameter, but there are a lot of bigger fibers with the fibers forming a network structure. Nanofibrillated cellulose tends to comprise individual fibrils, with nanoscale diameters and a narrow size distribution. In one embodiment, the cellulose nanofibrils have an average cross-sectional distance (longest dimension of a cross-section of the cellulose particle, perpendicular to the length) of at least 2, 4, or even 5 nanometers (nm) and at most 10, 20, 30, or even 50 nm; and an average length (longest dimension of the cellulose nanocrystal) of at least 50, 75, or even 100 nm and at most 150, 200, 250, 500, 750, or even 1000 nm. In another embodiment, the cellulose nanofibrils have an average cross-sectional distance (longest dimension of a cross-section of the cellulose particle, perpendicular to the length) of at least 100, 500, 1000, even 2000 nanometers (nm) and at most 1, 2, 5, 10, 50, 75, or even 100 micrometer, and an average length (longest dimension of the cellulose nanocrystal) of at least 0.5, 1, 2, 5, 10, 50, or even 100 micrometers and at most 150, 200, 250, 500, 750, or even 1000 micrometer. The cross-sectional morphology of the nanofibrils is typically square, but can be rectangular, or rounded. Typically, the cellulose nanofibrils have a high aspect ratio (ratio of height versus length). In one embodiment, the cellulose nanofibrils have an aspect ratio of 10 to 200, or even 100-150. The dimensions of the cellulose nanofibrils may be determined based on transmission electron microscopy (TEM), scanning electron microscopy (SEM), atomic force microscopy, or by other suitable means. Typically, the morphology is determined on dried samples. In one embodiment, the cellulose nanofibrils have an average surface area of at least 30, 40, 50, 70, or even 100 m²/g. In one embodiment, the cellulose nanofibrils have an average surface area of at most 100, 150, 200, 300, 400, or even 500 m²/g.

The zeta potential measures the potential difference existing between the surface of a solid particle immersed in a conducting liquid (e.g., water) and the bulk of the liquid of the cellulose nanofibril surface. The cellulose nanofibrils have a zeta potential higher (i.e., less negative) than −50, −45, −40, −35, −30, or even −25 mV based on dynamic light scattering.

The cellulose nanofibrils typically have a pH of less than 7.5, 7.0, 6.5, or even 6.0 and greater than 4.5, 5.0, or even 5.5 when measured at ambient conditions.

Cellulose nanofibrils may be obtained, for example, from CelluForce, Montreal, Canada; Melodea Ltd., Israel; American Process Inc., Atlanta, GA; Blue Goose Biorefineries Inc., Saskatoon, Canada; the USDA Forest Products Laboratory, Madison, WI via the University of Maine; and Weidmann Fiber Technology, Rapperswil, Switzerland.

In one embodiment disclosure, the cellulose nanofibrils are present in a ratio of at least 0.5:1; 0.75:1 or even 1:1 capture component to cellulose nanofibrils. In one embodiment disclosure, the cellulose nanofibrils are present in a ratio of at most 1:1, 1:1.25; 1:1.5; 1:2; or even 1:2.5 capture component to cellulose nanofibrils.

In one embodiment, the functionalized porous substrate is disposed on an absorbent layer. The absorbent layer or substrate pad, typically located below the functionalized porous substrate, opposing where the sample mixture is dispensed, draws the sample, through to the absorbent material below. The absorbent layer can be generated from any material capable of wicking fluid by way of capillary action, such as paper, cellulose, and cellulose derivatives such as cellulose acetate and nitrocellulose, fiberglass, cloth, cotton, polyester, polyolefin such as polyethylene, films of polyvinyl chloride, and the like. In one embodiment, the absorbent layer comprises a dried gel such as silica gel, agarose, dextran, or gelatin.

The selection of material for the absorbent layer is not critical and a variety of fibrous filter materials can be used, including one or more layers of the same or different materials, providing that the material selected is compatible with the target analyte and the assay reagents. Any conventionally employed absorbent material that is capable of drawing or wicking fluid through a porous membrane, such as for example, by capillary action, can be used in the present invention. The absorbent material should be capable of absorbing a volume of fluid test sample that is equivalent or greater than the total volume capacity of the material itself. Useful known materials include cotton, cotton linter, cellulose acetate fibers, polyester, polyolefin or other such materials. The absorbent material provides a means to collect the sample by providing uniform “suction” to deliver the sample from the well, through the reaction zone, and down into the absorbent material. Thus, the absorbent body also acts as a reservoir to hold the sample, and various reagents that are used when the assay is performed. Accordingly, when used in assays where relatively large volumes of fluid are used, the absorbent material should have high absorbent capacity so as to prevent or minimize the possibility of back-flow of sample and reagents from the absorbent body back into the reaction membrane.

The absorbent layer is placed on the opposite side of the porous substrate from where the sample matrix is added. In one embodiment, the absorbent layer is fixedly attached to the porous substrate, for example, by using adhesive.

### Method

In one embodiment, the multilayered substrate of the present disclosure is used in a downward or vertical flow assay, wherein the multilayered substrate is placed in a testing device comprising a test area. In the test area, the functionalized porous substrate faces outward with the absorbent layer there beneath and in vertical communication with the porous substrate.

Exemplary biological samples that can be sampled include: body fluids and tissue samples. Body fluids can include, amniotic fluid, aqueous humor, vitreous humor, bile, blood, cerebrospinal fluid, chyle, endolymph, perilymph, female ejaculate, lymph, mucus (including nasal drainage and phlegm), pericardial fluid, peritoneal fluid, pleural fluid, pus, rheum, saliva, sputum, synovial fluid, vaginal secretion, semen, blood, serum, or plasma. Tissue samples can include organ or tissue extract such as that taken from placenta, brain, eyes, pineal gland, pituitary gland, thyroid gland, parathyroid glands, thorax, heart, lung, esophagus, thymus gland, pleura, adrenal glands, appendix, gall bladder, urinary bladder, large intestine, small intestine, kidneys, liver, pancreas, spleen, stoma, ovaries, uterus, testis, skin, blood or buffy coat sample of blood. Additional examples of organs and tissues from any biological source are well known to a person of ordinary skill in the art and such embodiments are within the purview of the methods provided herein. In one example, swabbing from a nasal cavity, throat, or other body part is used for point of care testing.

In environmental testing and food safety testing, the test sample, may or may not be a liquid. For example, a water sample or liquid food sample may be the sample, or a swab may be used to wipe the surface of a food preparation surface, container, or high traffic area.

The reagent matrix is dispersed in liquid, if already not done so and the sample is added to the reagent matrix to form a test sample. In one embodiment, the reagent matrix is in a dried form and a liquid, such as a buffer and/or surfactant can be used to disperse the capture component reagents. Buffers can include those known in the art, especially biological buffers. A surfactant can be used to aid in the denaturation of the analyte and/or sample. Such surfactant include those known in the art. In one embodiment, the buffer is a multifunctional buffer as disclosed in U.S. Pat. No. 7,531,362 (Chan, herein incorporated by reference), which comprises a biological buffer to maintain the pH between about 7.0 to 10.0; at least one surfactant to reduce non-specific binding of assay reagents while simultaneously avoiding inhibition of a specific binding interaction; (3) a high molecular weight polymer as a dispersing and suspending reagent having a molecular weight in a range of from about 2×10⁶ to about 2×10⁶ D; a pH stabilizer to maintain the pH of the multifunctional buffer between about pH 7.0 to 10.0; an ionic salt to reduce the non-specific binding of antibodies, at least one preservative to reduce bacterial and microbial growth; and a calcium chelator to prevent a whole blood test sample from clotting.

The test sample including the sample and the reagent matrix in a liquid medium and optional surfactant is mixed to enable agglomeration of the analyte with the first and second capture components. The test sample is then disposed onto the test area of the testing device. Although not wanting to be limited by theory, it is believed that the agglomerated sample is captured on the surface of the functionalized porous substrate layer, while the reagents flow vertically or downwardly. After application of the test sample, it may be advantageous to include one or more washing steps, for example by adding a wash solution such as a buffer dropwise onto the testing area. It is believed that the capturing of the analyte and concentrating it on the surface of the functionalized porous substrate enables good detection of the analyte. Visual detection (in other words, by a human eye) or with an instrument (such as a spectrophotometer, a fluorescence detector, Geiger counter, etc.) may be used to determine if the analyte is present and/or the amount of analyte present in the sample.

In one embodiment, the testing device is one such that multiple analytes (for example 2, 3 or even more different analytes) are simultaneously detected. For example, a first capture component with a detection medium 1 may be used to detect analyte 1, which another first capture component with a detection medium 2 may be used to detect analyte 2. In one embodiment, detection medium 1 and 2 are both colorometric and the observed color is additive. In another embodiment, detection medium 1 and 2 use different detection mechanisms (e.g., colorometric and radio-labeled).

### Examples

Unless otherwise noted, all parts, percentages, ratios, etc. in the examples and the rest of the specification are by weight, and all reagents used in the examples were obtained, or are available, from general chemical suppliers such as, for example, Sigma-Aldrich Company, Saint Louis, Missouri, or may be synthesized by conventional methods.

### Materials and Methods

2-Hydroxyethyl methacrylate (HEMA), di(ethylene glycol) methyl ether methacrylate, (DEGMEMA), 2-ethoxyethyl methacrylate (2-EOEMA), poly(ethylene glycol) methyl ether methacrylate having a number-average molecular weight of 300 g/mol (PEG300-MA), glycidyl methacrylate (GMA), 4-aminobutanoic acid, L-phenylalanine, taurine, sulfanilic acid, caproic acid, and 4-morpholineethanesulfonic acid (MES) were obtained from Sigma-Aldrich (St. Louis, MO).

Phosphate buffered saline (PBS, 10×) was obtained from MilliporeSigma (Burlington, MA).

(1-Ethyl-3-(3-dimethylaminopropyl)carbodiimide hydrochloride) (EDC), BLOCKER casein in PBS (1% weight to volume), sucrose, TRITON X-100 surfactant, and N-hydroxysulfosuccinimide (sulfo-NHS) were obtained from Thermo Fisher Scientific (Waltham, MA).

2-Isocyanatoethyl methacrylate (IEM) was obtained from Showa Denko KK, Kanagawa, Japan.
Boric acid was obtained from VWR International (Radnor, PA).
Microfibrillated cellulose (MFC) was obtained under the trade designation “CELOVA M250R-G” from Weidmann Fiber Technology (Rapperswil, Switzerland) as a white gel with specifications of 2.9% MFC content, pH 8, specific surface area of 234 m²/g, and particle length D₅₀ of 9 micrometers reported by the manufacturer.
Influenza A Nucleoprotein (NP), product no. 9593, was obtained from Meridian Bioscience, Memphis, TN.
SARS-CoV-2 Nucleoprotein (NP), product no. RP-87665 was obtained from ThermoFisher, Waltham, MA.
Unless otherwise noted, deionized, 18 megaohm water from a MILLI-Q water purification system (EMD Millipore, Billerica, MA) was used.

### Preparation of Influenza A Monoclonal Antibody Functionalized Nanocellulose Beads—Type A

NANOACT blue colored, nanocellulose beads (catalog #BLIBCA005, carboxylic acid functionalized, 318 nm diameter, 1.05 wt-% suspension in water) were obtained from Asahi Kasei Corporation, Tokyo, Japan. An aliquot of the NANOACT suspension (60 microliters) was added to a pre-weighed 15 mL conical centrifuge tube followed by the sequential addition of 540 microliters of MES (100 millimolar aqueous solution, pH 6), 7.5 microliters of EDC (4 wt.-% aqueous solution), and 15 microliters of sulfo-NHS (4 wt.-% aqueous solution). The tube was maintained at room temperature for 15 minutes and then centrifuged (5000 g) at 20° C. for 20 minutes using a benchtop centrifuge. The resulting supernatant liquid was removed from the tube using a micro-pipette. An aliquot of MES (600 microliters of 100 mM aqueous solution, pH 6) was added to the tube and the suspension was sonicated using a probe sonicator for 10 seconds in pulse mode (set at 3.2 seconds on and 0.5 seconds off). The antibody (60 micrograms of Monoclonal Antibody (mAb) to Influenza A (NP)), product no. C01736M obtained from Meridian Bioscience) was added to the resuspended beads and the tube was vortexed for 5-10 seconds using a Vortex Genie-2 at maximum speed (Scientific Industries, Inc., Bohemia, NY). The suspension was then incubated for 120 minutes at 37° C. in a temperature-controlled chamber. Blocker casein in PBS (7200 microliters) was added and the tube was vortexed for 5-10 seconds and then incubated at 37° C. for 60 minutes. The tube was removed from the temperature-controlled chamber and centrifuged (5000 g) at 20° C. for 20 minutes using a benchtop centrifuge. The resulting supernatant liquid was removed by decanting and then 7200 microliters of boric acid (50 mM aqueous solution, pH 10) was added to the tube. The suspension was sonicated using a probe sonicator for 10 seconds in pulse mode (set at 3.2 seconds on and 0.5 seconds off) followed by centrifuging the tube (5000 g) at 20° C. for 20 minutes. Supernatant was removed by decanting and the resulting conjugated beads were resuspended with a 33 mM aqueous boric acid solution (pH 9.2) that also contained blocker casein (0.2 wt.-%) and sucrose (15 wt.-%). Enough solution was added so that the total weight of the suspension was 1579 mg. This resulted in a 0.038 wt.-% stock suspension of antibody conjugated beads. The suspension was sonicated using a probe sonicator for 10 seconds in pulse mode (set at 3.2 seconds on and 0.5 seconds off). The tubes were stored at 4° C. and used within 2-3 days. Alternatively, the suspension of conjugated beads was aliquoted to 1.5 mL Eppendorf tubes and freeze-dried. The freeze-dried samples were sealed in foil pouches. Each pouch contained two 1 g silica gel desiccant packs (product no. S-3902, obtained from ULINE Company, Pleasant Prairie, WI) and was stored at room temperature.

Preparation of Influenza A Monoclonal Antibody (mAb) Functionalized Nanocellulose Beads—Type B

The same procedure as reported for the “Preparation of Monoclonal Antibody Functionalized Nanocellulose Beads—Type A” was followed with the exception that the antibody conjugated to the beads was Monoclonal Antibody (mAb) to Influenza A nucleocapsid protein, (product no. C01737M, obtained from Meridian Bioscience)

Preparation of Influenza A Monoclonal Antibody (mAb) Functionalized Nanocellulose Beads—Type C

The same procedure as reported for the “Preparation of Monoclonal Antibody Functionalized Nanocellulose Beads—Type A” was followed with the exception that the beads were replaced with NANOACT red colored, nanocellulose beads (product no. RE1CCA001, carboxylic acid functionalized, 331 nm diameter, 1.04 wt.-% suspension in water, obtained from Asahi Kasei Corporation).

Preparation of Influenza A Monoclonal Antibody (mAb) Functionalized Nanocellulose Beads—Type D

The same procedure as reported for the “Preparation of Monoclonal Antibody Functionalized Nanocellulose Beads—Type B” was followed with the exception that the beads were replaced with NANOACT red colored, nanocellulose beads (product no. RE1CCA001).

Preparation of SARS-CoV-2 Monoclonal Antibody (mAb) Functionalized Nanocellulose Beads—Type E.

The same procedure as reported for the “Preparation of Monoclonal Antibody Functionalized Nanocellulose Beads—Type A” was followed with the exception that the antibody conjugated to the beads was Monoclonal Antibody (mAb) to SARS-CoV-2 Nucleocapsid (product no. 9547, obtained from Meridian Bioscience, Cincinnati, OH).

Preparation of SARS-CoV-2 Monoclonal Antibody (mAb) Functionalized Nanocellulose Beads—Type F.

The same procedure as reported for the “Preparation of Monoclonal Antibody Functionalized Nanocellulose Beads—Type A” was followed with the exception that the antibody conjugated to the beads was Monoclonal Antibody (mAb) to SARS-CoV-2 Nucleocapsid (product no. 9548, obtained from Meridian Bioscience).

Preparation of SARS-CoV-2 Monoclonal Antibody (mAb) Functionalized Nanocellulose Beads—Type G.

The same procedure as reported for the “Preparation of Monoclonal Antibody Functionalized Nanocellulose Beads—Type C” was followed with the exception that the antibody conjugated to the beads was Monoclonal Antibody (mAb) to SARS-CoV-2 Nucleocapsid (product no. 9547, obtained from Meridian Bioscience).

Preparation of SARS-CoV-2 Monoclonal Antibody (mAb) Functionalized Nanocellulose Beads—Type H.

The same procedure as reported for the “Preparation of Monoclonal Antibody Functionalized Nanocellulose Beads—Type C” was followed with the exception that the antibody conjugated to the beads was Monoclonal Antibody (mAb) to SARS-CoV-2 Nucleocapsid (product no. 9548, obtained from Meridian Bioscience).

Preparation of SARS-CoV-2 Monoclonal Antibody (mAb) Functionalized Latex Beads—Type I.

The same procedure as reported for the “Preparation of SARS-CoV-2 Monoclonal Antibody (mAb) Functionalized Nanocellulose Beads—Type E” was followed with the exception that the nanocellulose beads were replaced with red colored latex beads (catalog #DCCR004, 1 micrometer diameter, obtained from Bangs Laboratories, Fishers, IN).

Preparation of SARS-CoV-2 Monoclonal Antibody (mAb) Functionalized Latex Beads—Type J.

The same procedure as reported for the “Preparation of SARS-CoV-2 Monoclonal Antibody (mAb) Functionalized Nanocellulose Beads—Type F” was followed with the exception that nanocellulose beads were replaced with red colored latex beads (catalog #DCCR005, 5 micrometer diameter, obtained from Bangs Laboratories).

### Monomer Preparations:

### Monomer A.

### 4-[2-(2-Methylprop-2-enoyloxy)ethylcarbamoylamino]butanoic acid, Sodium Salt (IEM-GABA, Sodium Salt)

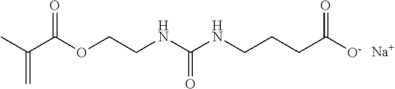 [SMILES: C=C(C)C(=O)OCCNC(=O)NCCCC(=O)[O-] ; [Na+]]

4-Aminobutanoic acid (2.06 g, 0.02 mol) was added to a 100 mL round bottom flask. An aqueous solution of sodium hydroxide (1.0 N, 20 mL) was added to the flask and the resulting mixture was stirred until the solids dissolved. The flask was then placed in an ice-water bath and stirred for 15 minutes. IEM (3.1 g, 0.02 mol) was added by syringe and the reaction was stirred for 30 minutes with the flask continuously maintained in the ice-water bath. The cooling bath was then removed and the reaction was allowed to warm to room temperature over a period of 30 minutes. A colorless precipitate was filtered from the reaction mixture. The pH of the filtrate was adjusted to about 7 by the addition of a few drops of a concentrated hydrochloric acid solution. ¹H-NMR of an aliquot of the filtrate confirmed the formation of 4-[2-(2-methylprop-2-enoyloxy)ethylcarbamoylamino]butanoic acid, sodium salt. ¹H-NMR (D₂O, 500 MHz) δ 1.57 (t, 2H), 1.78 (s, 3H), 2.05 (t, 2H), 2.95 (m, 2H), 3.31 (m, 2H), 4.08 (m, 2H), 5.58 (s 1H), 5.99 (s, 1H).

### Monomer B.

### 2-[2-(2-Methylprop-2-enoyloxy)ethylcarbamoylamino]-3-phenylpropanoic acid, Sodium Salt

### (IEM-Phenylalanine, Sodium Salt)

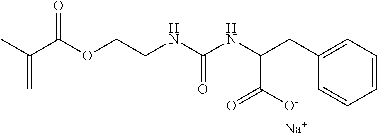 [SMILES: C=C(C)C(=O)OCCNC(=O)NC(Cc1ccccc1)C(=O)[O-] ; [Na+]]

L-Phenylalanine (3.3 g, 0.02 mol) was charged to a 100 mL round bottom flask. An aqueous solution of sodium hydroxide (1.0 N, 20 mL) was added to the flask and the resulting mixture was stirred until the solids dissolved. The flask was then placed in an ice-water bath and stirred for 15 minutes. IEM (3.1 g, 0.02 mol) was added by syringe and the reaction was stirred for 30 minutes with the flask continuously maintained in the ice-water bath. The cooling bath was then removed, and the reaction was allowed to warm to room temperature over a period of 30 minutes. A colorless precipitate was filtered from the reaction mixture. The pH of the reaction was adjusted to about 7 by the addition of a few drops of a concentrated hydrochloric acid solution. ¹H-NMR of an aliquot of the filtrate confirmed the formation of 2-[2-(2-methylprop-2-enoyloxy)ethylcarbamoylamino]-3-phenylpropanoic acid, sodium salt. ¹H-NMR (D₂O, 500 MHz) δ 1.74 (br. s, 3H), 2.73 (m, 1H), 2.99 (m, 1H), 3.13 (m, 1H), 3.26 (m 1H), 3.90 (m, 2H), 4.17 (m, 1H), 5.54 (m, 1H), 5.95 (m, 1H), 7.09 and 7.15 (m, 5H).

### Monomer C.

### 2-[2-(2-Methylprop-2-enoyloxy)ethylcarbamoylamino]ethanesulfonic acid, Sodium Salt

### (IEM-Taurine, Sodium Salt)

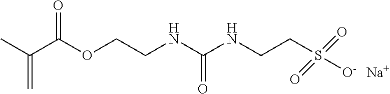 [SMILES: C=C(C)C(=O)OCCNC(=O)NCCS(=O)(=O)[O-] ; [Na+]]

Taurine (2.50 g, 0.02 mol) was added to a 100 mL round bottom flask. An aqueous solution of sodium hydroxide solution (1.0 N, 20 mL) was added to the flask and the resulting mixture was stirred until the solids dissolved. The flask was then placed in an ice-water bath and stirred for 15 minutes. IEM (3.1 g, 0.02 mol) was added by syringe and the reaction was stirred for 30 minutes with the flask continuously maintained in the ice-water bath. The cooling bath was then removed, and the reaction was allowed to warm to room temperature over a period of 30 minutes. A colorless precipitate was filtered from the reaction mixture. The pH of the reaction was adjusted to about 7 by the addition of a few drops of a concentrated hydrochloric acid solution. ¹H-NMR of an aliquot of the filtrate confirmed the formation of 2-[2-(2-methylprop-2-enoyloxy)ethylcarbamoylamino]ethanesulfonic acid, sodium salt. ¹H-NMR (D₂O, 500 MHz) δ 1.75 (s, 3H), 2.88 (t, 2H), 3.28 (t, 2H) 3.32 (t, 2H), 4.06 (t, 2H), 5.56 (m, 1H), 5.97 (s, 1H).

### Monomer D.

### 4-[2-(2-methylprop-2-enoyloxy)ethylcarbamoylamino]benzenesulfonic acid, Sodium Salt

### (IEM-Sulfanilic Acid, Sodium Salt)

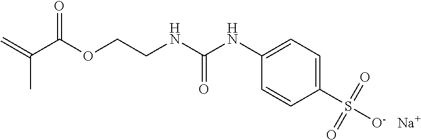 [SMILES: C=C(C)C(=O)OCCNC(=O)Nc1ccc(S(=O)(=O)[O-])cc1 ; [Na+]]

Sulfanilic acid (69.30 g, 0.4 mol) was added to a 1000 mL round bottom flask. An aqueous solution of sodium hydroxide solution (1.0 N, 415 mL) was added to the flask and the resulting mixture was stirred until the solids dissolved. The flask was then placed in an ice-water bath and stirred for 30 minutes. IEM (62.06 g, 0.4 mol) was added drop wise, and the reaction was stirred for 30 minutes with the flask continuously maintained in the ice-water bath. The cooling bath was then removed, and the reaction was allowed to warm to room temperature over a period of 30 minutes. A colorless precipitate was filtered from the reaction mixture. The pH of the reaction was adjusted to about 7 by the addition of a few drops of a concentrated hydrochloric acid solution. ¹H-NMR of an aliquot of the filtrate confirmed the formation of 4-[2-(2-methylprop-2-enoyloxy)ethylcarbamoylamino]benzenesulfonic acid, sodium salt. ¹H-NMR (D₂O, 500 MHz) δ 1.76 (s, 3H), 3.39 (s, 2H), 4.12 (s, 2H), 5.55 (s, 1H), 5.98 (s, 1H), 7.25 (d, J=8.68 Hz, 2H), 7.57-7.60 (m, 2H).

### Monomer E.

### 6-[2-(2-methylprop-2-enoyloxy)ethylcarbamoylamino]hexanoic acid, Sodium Salt

### (IEM-Caproic Acid, Sodium Salt)

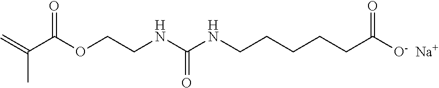 [SMILES: C=C(C)C(=O)OCCNC(=O)NCCCCCC(=O)[O-] ; [Na+]]

Monomer E was prepared according to the procedure described in Prep. Ex. 15 of U.S. Pat. No. 9,958,364 (Rasmussen, et al.).

### Monomer F.

### [2-(2-Methylprop-2-enoyloxy)ethylcarbamoylamino]methylphosphonic acid

### (IEM-Phosphonic Acid)

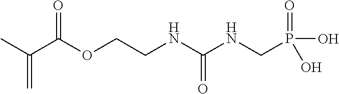 [SMILES: C=C(C)C(=O)OCCNC(=O)NCP(=O)(O)O]

Monomer F was prepared according to the procedure described for the preparation of Monomer C in PCT Patent Application WO2022013641 (Colak Atan, et al.).

### Grafting Solutions

Individual aqueous grafting solutions were prepared with the monomer compositions reported in Table 1. All components in the grafting solutions are reported in weight percent (wt.-%).

TABLE 1

| Grafting | | | |
|---|---|---|---|
| Solution | Monomer Component | Monomer Component | Water |
| A | 2-EOEMA (4.5 wt.-%) | none | 95.5 wt.-% |
| B | IEM-GABA (10.0 wt.-%) | GMA (2.0 wt.-%) | 88.0 wt.-% |
| C | IEM-Taurine (10.8 wt.-%) | GMA (2.0 wt.-%) | 87.2 wt.-% |
| D | IEM-Phenylalanine (12.2 wt.-%) | GMA (2.0 wt.-%) | 85.8 wt.-% |
| E | Methacrylic acid (3.1 wt.-%) | GMA (2.0 wt.-%) | 94.9 wt.-% |
| F | IEM-Sulfanilic acid (12.5 wt.-%) | GMA (2.0 wt.-%) | 85.5 wt.-% |
| G | IEM-Caproic acid (11.0 wt.-%) | GMA (2.0 wt.-%) | 87.0 wt.-% |
| H | IEM-Phosphonic acid (11.1 wt.-%) | GMA (2.0 wt.-%) | 86.9 wt.-% |
| I | HEMA (3.7 wt.-%) | none | 96.3 wt.-% |
| J | DEGMEMA (5.3 wt.-%) | none | 94.7 wt.-% |
| K | PEG300-MA (8.4 wt.-%) | none | 91.6 wt.-% |

### Example 1. Preparation of Functionalized Nonwoven Substrate A (FNW-A)

A melt-blown polypropylene nonwoven web (white in color and having an effective fiber diameter of about 12 micrometers, basis weight of about 200 grams per square meter (gsm), solidity of about 10%, and calculated average pore size of 35.5 micrometers) was grafted with nitrogen purged Grafting Solution A. A sample of the nonwoven web (17.8 cm by 22.9 cm) was placed in a glove box and purged of air under a nitrogen atmosphere. Once the oxygen levels reached less than 20 ppm, the nonwoven substrate was inserted into a plastic bag and the bag was sealed.

Grafting solution A (2-EOEMA, 100 grams) was added to a glass jar. The jar was capped and shaken by hand to mix the contents. The jar was then opened and the solution was sparged with nitrogen for at least 2 minutes to remove any dissolved oxygen from the solution. The jar was re-capped and transferred into the oxygen depleted glovebox. The jar lid was then removed to flush any residual air from the jar headspace.

The sealed bag containing nonwoven sample was removed from the glove box and irradiated with an electron beam (Electrocure, Energy Sciences Inc, Wilmington, MA) at an accelerating voltage of 300 kV to a dose of 6 Mrad. The bag containing the irradiated nonwoven sample was then returned to the glove box and purged of air as described above. Grafting Solution A (100 g) was added to the plastic bag containing the nonwoven sample. The bag was sealed and the solution was distributed through the nonwoven sample using a hand roller so that the nonwoven sample was uniformly covered with the solution. The nonwoven sample was maintained flat in the sealed bag for 3 hours. The bag was removed from the glove box and then opened to allow atmospheric oxygen to quench the reaction. The resulting polymer-grafted nonwoven sample was removed from the bag and boiled in deionized water for one hour. The sample was removed from the water bath and air dried at room temperature for 24-72 hours. The resulting dried polymer-grafted nonwoven sample was labeled as Functionalized Nonwoven Substrate A (FNW-A). The dried functionalized nonwoven substrate sample was white in color.

The amount (g) of monomer grafted to the nonwoven sample as grafted polymer was measured gravimetrically by weighing the nonwoven sample before and after the grafting procedure. The weight after the grafting procedure was measured following the final drying step. The percent weight gain from the grafting procedure was determined according to Equation 2. For FNW-A, the percent weight gain from the grafting procedure was 57%.
“Average pore size” (also known as average pore diameter) was calculated according to Equation 1.

$$Average pore size = {d}_{f} ( {( 2 α / π )}^{( - 1 / 2 )} - 1 ) \qquad Equation 1$$

where “d_{f}” is the arithmetic median fiber diameter, and “α” is the web solidity.

$$\% Weight Gain = [ ( Mass of Dry FNW - Initial Mass of Nonwoven ) \text{} / \text{} ( Initial Mass of Nonwoven ) ] \times 100 \qquad Equation 2$$

### Example 2. Preparation of Functionalized Nonwoven Substrates B-K (FNW-B-FNW-K)

The same procedure as described in Example 1 was followed with the exception that Grafting Solution A was replaced with a single grafting solution selected from Grafting Solutions B-K. The properties of the FNWs A-K are summarized in Table 3. All of the dried functionalized nonwoven substrates FNW-B-FNW-K were white in color.

TABLE 3

| | Monomers in Grafting Solution | Percent Weight |
|---|---|---|
| | Used to Prepare Functionalized | Gain from |
| FNW | Nonwoven Substrates | Grafting Procedure |
| FNW-A | 2-EOEMA | 57% |
| FNW-B | IEM-GABA + GMA | 44% |
| FNW-C | IEM-Taurine + GMA | 28% |
| FNW-D | IEM-Phenylalanine + GMA | 25% |
| FNW-E | Methacrylic acid + GMA | 61% |
| FNW-F | IEM-Sulfanilic acid + GMA | 70% |
| FNW-G | IEM-Caproic acid + GMA | 43% |
| FNW-H | IEM-Phosphonic acid + GMA | 50% |
| FNW-I | HEMA | 51% |
| FNW-J | DEGMEMA | 42% |
| FNW-K | PEG300-MA | 56% |

### Example 3. Preparation of Devices AA-KK

A vertical flow detection device was prepared. The device consisted of a sealed plastic housing with an internal cavity (external device dimensions: 10 cm (length)×7.5 cm (width)×14 mm (depth) that was prepared by 3D-printing using ACCURA 25 plastic and a 3D Systems PROJET 7000 printer (3D Systems, Rock Hill, SC). The device housing was prepared from two halves (i.e., upper and lower housing sections connected together with latches). The lower housing section of the device contained an internal cavity (dimensions of 51 mm (length)×13 mm (width)×1.5 mm (depth). The upper housing section of the device contained 2 circular openings (each opening 4 mm in diameter). The openings were positioned to be aligned with the cavity section of the lower housing and were spaced apart by 20 mm in the lengthwise direction. The absorbent pads for the device were 50 mm by 13 mm sections cut from WHATMAN Grade GB003 cellulose blotting paper (0.8 mm thick) (obtained from Cytvia, Marlborough, MA). The porous substrate layer of the device was a 50 mm by 13 mm section cut from a single functionalized nonwoven substrate selected from FNW-A-FNW-K. A stack of three absorbent pads was placed in the cavity of the lower housing and a single section of the selected functionalized nonwoven substrate was placed on top of the stack of absorbent pads. The upper and lower housing sections were then mated and secured using magnetic closures to form the finished device. In the finished device, the internal facing surface of the upper housing section pressed against the surface of the functionalized nonwoven layer in the stack. The two openings in the upper housing and the functionalized nonwoven surface formed two wells in the device that were used for sample delivery and assay result detection. The constructions of Devices AA-KK are summarized in Table 4.

### Example 4. Detection of Influenza A N-Protein (NP) Using Devices AA-KK

Analyte test samples were prepared by adding an aliquot (300 microliters) of PBS (diluted to 1×PBS, pH 7.4) that contained TRITON X-100 surfactant (1% by volume) and either 20 nM, 2 nM, 0.5 nM, or 0.2 nM of Influenza A NP to a microcentrifuge tube that contained Influenza A mAb Functionalized Nanocellulose Beads-Type A (0.00475 mg) and Influenza A mAb Functionalized Nanocellulose Beads-Type B (0.00475 mg). The resulting suspension was mixed by inverting the microcentrifuge tube several times. The suspension was maintained for 2 minutes and then added to a device. Control samples were also prepared using the procedure with the exception that the Influenza A NP component was omitted from the control samples. A single analyte test sample (300 microliters) was added by pipette to the first well opening (i.e., analyte test well) of a device (selected from Devices AA-FF) and the corresponding control sample (300 microliters) was added by pipette to the second well opening (i.e., control test well) of the selected device. About 30 seconds after administering the sample, a 100 microliter aliquot of a 0.1M PBS solution that contained 300 mM NaCl and 1% (by volume) Triton X-100 aqueous surfactant was added by pipette to each well.

The functionalized nonwoven layer was removed from each device and placed in a portable photo booth. An image of the functionalized nonwoven surface from each well of a device was taken using a Canon EOS Rebel T3i digital camera (Canon USA, Melville, NY). The surface of the nonwoven layer to which the analyte test sample was added was positioned to face the camera. The images were analyzed using the ImageJ software program (National Institutes of Health, Bethesda, MD). The images were converted into 8-bit images (grayscale) and inverted. The image pixel intensity for each well of the device was quantified using the analyze and measure functions of the program. Normalized pixel intensity was obtained for the first well image (i.e., sample spiked with Influenza A NP) by subtracting the pixel intensity value of the second well image (i.e., pixel intensity of the control sample) from the pixel intensity value of the first well image. A normalized pixel intensity value for the first well image of greater than 1 was determined to be a positive result for Influenza A NP in the analyte test sample. A normalized pixel intensity value for the first well image of less than or equal 1 was determined to be a negative result for Influenza A NP in the analyte test sample. In Table 4, the minimum concentration of Influenza A NP in an analyte test sample at which a positive result was detected is reported.

TABLE 4

| | Functionalized | Minimum Concentration of |
|---|---|---|
| | Nonwoven Layer of | Influenza A NP with a Positive |
| Device | Device (Table 3) | Result Detected (nM) |
| Device AA | FNW-A | 20 |
| Device BB | FNW-B | 20 |
| Device CC | FNW-C | 2 |
| Device DD | FNW-D | 0.5 |
| Device EE | FNW-E | 2 |
| Device FF | FNW-F | 2 |
| Device GG | FNW-G | 20 |
| Device HH | FNW-H | No Positive Result Observed |
| Device II | FNW-I | 20 |
| Device JJ | FNW-J | No Positive Result Observed |
| Device KK | FNW-K | No Positive Result Observed |

### Example 5. Detection of SARS-CoV-2 NP Using Devices AA-KK

The same procedure of Example 4 was followed using different analyte test samples and control samples. Analyte test samples were prepared by adding an aliquot (300 microliters) of PBS (diluted to 1×PBS, pH 7.4) that contained TRITON X-100 surfactant (1% by volume) and either 20 nM, 2 nM, 0.5 nM, or 0.2 nM of SARS-CoV-2 NP to a microcentrifuge tube that contained SARS-CoV-2 mAb Functionalized Nanocellulose Beads—Type E (0.00475 mg) and SARS-CoV-2 mAb Functionalized Nanocellulose Beads—Type F (0.00475 mg). In some analyte test samples, microfibrillated cellulose (0.015 mg) was added to the microcentrifuge tube. The suspension was mixed and then maintained for 2 minutes before adding to a device selected from Devices AA-KK. The Control Samples were prepared using the same procedure with the exception that the SARS-CoV-2 NP component was omitted from the control samples.

In Tables 5 and 6, the minimum concentration of SARS-CoV-2 NP in an analyte test sample at which a positive result was detected is reported.

TABLE 5

| | | | Minimum |
|---|---|---|---|
| | Functionalized | MFC included | Concentration of |
| | Nonwoven | in the Analyte | SARS-CoV-2 NP |
| | Layer of Device | and Control | with a Positive Result |
| Device | (Table 3) | Samples | Detected (nM) |
| Device AA | FNW-A | No | 0.5 |
| Device BB | FNW-B | No | 2 |
| Device CC | FNW-C | No | 0.5 |
| Device DD | FNW-D | No | 0.5 |
| Device EE | FNW-E | No | 0.5 |
| Device FF | FNW-F | No | 2 |
| Device GG | FNW-G | No | 0.5 |
| Device HH | FNW-H | No | 2 |
| Device II | FNW-I | No | 2 |
| Device JJ | FNW-J | No | 2 |
| Device KK | FNW-K | No | No Positive |
| | | | Result Detected |

TABLE 6

| | | | Minimum |
|---|---|---|---|
| | Functionalized | MFC included | Concentration of |
| | Nonwoven | in the Analyte | SARS-CoV-2 NP |
| | Layer of Device | and Control | with a Positive Result |
| Device | (Table 3) | Samples | Detected (nM) |
| Device AA | FNW-A | Yes | 0.5 |
| Device BB | FNW-B | Yes | 2 |
| Device CC | FNW-C | Yes | 0.2 |
| Device DD | FNW-D | Yes | 0.5 |
| Device EE | FNW-E | Yes | 0.2 |
| Device FF | FNW-F | Yes | 20 |
| Device GG | FNW-G | Yes | 0.5 |
| Device HH | FNW-H | Yes | 0.5 |
| Device II | FNW-I | Yes | 2 |
| Device JJ | FNW-J | Yes | 2 |
| Device KK | FNW-K | Yes | 2 |

### Example 6. Detection of SARS-CoV-2 NPn Using mAb Functionalized Latex Beads

The same procedure of Example 4 was followed using different analyte test and control samples. Analyte test samples were prepared by adding an aliquot (300 microliters) of PBS (diluted to 1×PBS, pH 7.4) that contained TRITON X-100 surfactant (1% by volume) and either 20 nM, 2 nM, or 0.5 nM of SARS-CoV-2 NP to a microcentrifuge tube that contained SARS-CoV-2 mAb Functionalized Latex Beads—Type I (0.00475 mg) and SARS-CoV-2 mAb Functionalized Latex Beads—Type J (0.00475 mg). The suspension was mixed and then maintained for 2 minutes before adding to a selected device. The Control Samples were prepared using the same procedure with the exception that the SARS-CoV-2 NP component was omitted from the control samples. In Table 7, the minimum concentration of SARS-CoV-2 NP in an analyte test sample at which a positive result was detected is reported.

TABLE 7

| Functionalized | Minimum Concentration of |
|---|---|
| Nonwoven Layer of | SARS-CoV-2 NP with a Positive |
| Device (Table 3) | Result Detected (nM) |
| FNW-A | 20 |
| FNW-C | 20 |
| FNW-D | 2 |
| FNW-F | No Positive Result Detected |

### Example 7. Simultaneous Detection of SARS-CoV-2 NP and Influenza A NP Using mAb Functionalized Nanocellulose Beads

The same procedure of Example 4 was followed using Vertical Flow Devices AA with different analyte test and control samples. Six analyte test samples were prepared by first preparing six individual aliquot samples (300 microliters) that contained PBS (diluted to 1×PBS, pH 7.4), TRITON X-100 surfactant (1% by volume), and varying concentrations of SARS-CoV-2 NP and/or Influenza A NP. Two of the aliquot samples contained only SARS-CoV-2 NP (20 or 2 nM). Two of the aliquot samples contained only Influenza A NP (20 or 2 nM). One aliquot sample contained both SARS-CoV-2 NP (20 nM) and Influenza A NP (20 nM). One aliquot sample contained both SARS-CoV-2 NP (2 nM) and Influenza A NP (2 nM). Each aliquot sample was individually added to a separate microcentrifuge tube (i.e., a single aliquot sample per microcentrifuge tube) that contained Influenza A mAb Functionalized Nanocellulose Beads—Type A (0.002375 mg), Influenza A mAb Functionalized Nanocellulose Beads—Type B (0.002375 mg), SARS-CoV-2 mAb Functionalized Nanocellulose Beads—Type G (0.002375 mg), and SARS-CoV-2 mAb Functionalized Nanocellulose Beads—Type H (0.002375 mg). Each resulting test sample was mixed and then maintained for 2 minutes before adding to a vertical flow device of type AA (Table 4). Control Samples were also prepared using the same procedure with the exception that both SARS-CoV-2 NP and Influenza A NP were omitted from the control samples.

The wells of the devices were visually examined for a color change. When only SARS-CoV-2 NP was included in the analyte test sample, the bottom surface of the analyte test well was red in color. When only Influenza A NP was included in the analyte test sample, the bottom surface of the analyte test well was blue in color. When both SARS-CoV-2 NP and Influenza A NP were included in the analyte test sample, the bottom surface of the analyte test well was purple in color. The control wells of all of the devices did not show a color change (i.e., the white color of the functionalized nonwoven surface of the control well was generally unchanged from before addition of the control sample). Analyte test wells with a red color were determined to be positive for the presence of SARS-CoV-2 NP and negative for the presence of Influenza A NP. Analyte test wells with a blue color were determined to be positive for the presence of Influenza A NP and negative for the presence of SARS-CoV-2 NP. Analyte test wells with a purple color were determined to be positive for the presence of both SARS-CoV-2 NP and Influenza A NP. Analyte test wells in which a color change was not observed (i.e., original white color maintained) were determined to be negative for the presence of SARS-CoV-2 NP and Influenza A NP. The results are summarized in Table 8.

TABLE 8

| SARS-CoV-2 | Influenza A | Color Observed in | Detection Result for Analyte |
|---|---|---|---|
| NP in Analyte | NP in Analyte | Analyte Test Well | Sample Based on Color Observed |
| (nM) | (nM) | of the Device | in the Analyte Test Well |
| 20 | 0 | Red | Positive for SARS-CoV-2 NP, |
| | | | Negative for Influenza A NP |
| 2 | 0 | Light Red | Positive for SARS-CoV-2 NP, |
| | | | Negative for Influenza A NP |
| 0 | 20 | Blue | Negative for SARS-CoV-2 NP, |
| | | | Positive for Influenza A NP |
| 0 | 2 | Light Blue | Negative for SARS-CoV-2 NP, |
| | | | Positive for Influenza A NP |
| 20 | 20 | Purple | Positive for SARS-CoV-2 NP, |
| | | | Positive for Influenza A NP |
| 2 | 2 | Purple | Positive for SARS-CoV-2 NP, |
| | | | Positive for Influenza A NP |

Foreseeable modifications and alterations of this invention will be apparent to those skilled in the art without departing from the scope and spirit of this invention. This invention should not be restricted to the embodiments that are set forth in this application for illustrative purposes. To the extent that there is any conflict or discrepancy between this specification as written and the disclosure in any document mentioned or incorporated by reference herein, this specification as written will prevail.

1. A multilayered article comprising:
(a) a porous substrate comprising a plurality of grafted groups to the porous substrate, wherein the plurality of grafted groups are derived from at least one of (i) an acidic monomer comprising at least one acidic group or salts thereof and a monovalent ethylenically unsaturated group selected from a (meth)acrylate or a (meth)acrylamide; (ii) a neutral hydrophilic monomer comprising at least one —O—CH₂CH₂— group and a monovalent ethylenically unsaturated group selected from a (meth)acrylate or a (meth)acrylamide; or (iii) combinations thereof; and
(b) an absorbent substrate thereon.

2. The multilayered article according to claim 1, wherein the porous substrate is a microporous membrane, optionally wherein the microporous membrane has a mean flow pore size of at least 0.1 micrometers and at most 100 micrometers.

3. The multilayered article according to claim 1, wherein the porous substrate is a non-woven substrate, optionally, wherein the nonwoven substrate comprises a plurality of microfibers having an effective fiber diameter of at least 0.5 and at most 16 micrometers.

4. The multilayered article according to claim 3, wherein the nonwoven substrate comprises a plurality of polyolefin fibers, polyamide fibers, polyester fibers, fiberglass, and combinations thereof.

5. The multilayered article according to claim 1, wherein the at least one acid group or salt thereof is selected from a carboxy, phosphono, phosphato, sulfono, sulfato, boronato, and combinations thereof.

6. The multilayered article according to claim 1, wherein the acidic monomer comprises a catenated heteroatom-containing hydrocarbon group.

7. The multilayered article according to claim 1, wherein the acidic monomer comprises at least one hydrogen bonding moiety, optionally, wherein the at least one hydrogen bonding moiety is selected from carbonylimino, thiocarbonylimino, iminocarbonylimino, iminothiocarbonylimino, oxycarbonylimino, oxythiocarbonylimino, and combinations thereof.

8. The multilayered article according to claim 1, wherein the second neutral hydrophilic monomer comprises no more than six —O—CH₂CH₂— groups.

9. The multilayered article according to claim 1, wherein the monomer is according to formula (IA) or (IB): 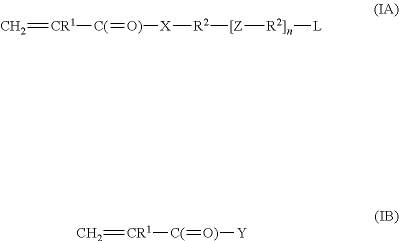
wherein
R¹ is selected from hydrogen, alkyl, cycloalkyl, aryl, and combinations thereof,
each R² is independently selected from hydrocarbylene, heterohydrocarbylene, and combinations thereof,
X is —O— or —NR³—, where R³ is selected from hydrogen, hydrocarbyl, heterohydrocarbyl, and combinations thereof,
Y is —OH;
Z is heterohydrocarbylene comprising at least one hydrogen bond donor, at least one hydrogen bond acceptor, or a combination thereof,
n is an integer of 0 or 1; and
L is a heteroatom-containing group comprising at least one monovalent ligand functional group selected from acidic groups, salt of acidic group.

10. The multilayered article according to claim 1, wherein the monomer is according to formula (V): 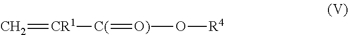
wherein
R¹ is selected from hydrogen, alkyl, cycloalkyl, aryl, and combinations thereof, and
R⁴ is H, or an oxy-hydrocarbyl,
wherein the monomer according to Formula (V) comprises no more than six —O—CH₂CH₂— groups.

11. The multilayered article according to claim 1, wherein the acidic monomer is at least one of the following compounds: 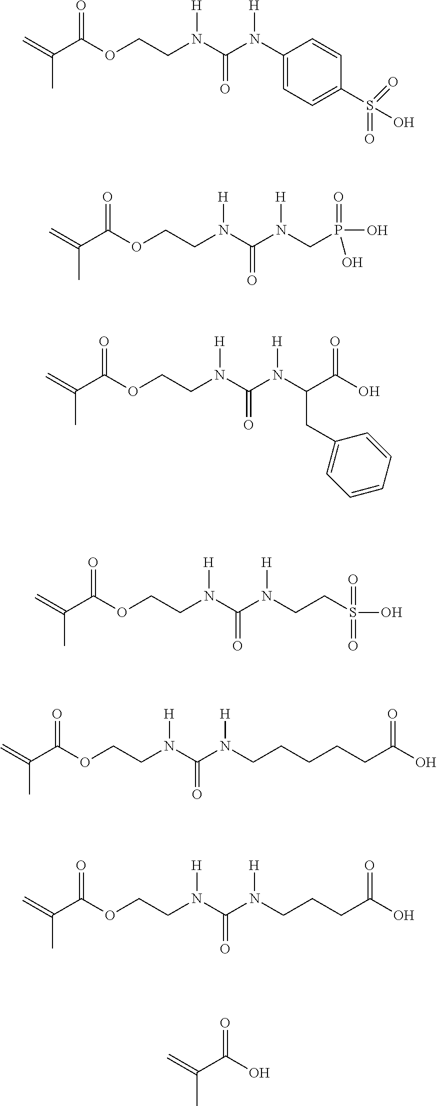
or salts thereof.

12. The multilayered article according to claim 1, wherein the neutral hydrophilic monomer is at least one of the following compounds: 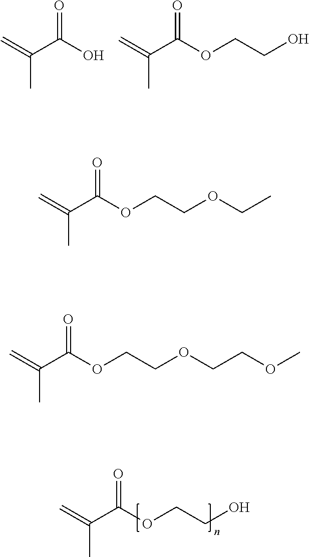
where n is 5 or 6.

13. The multilayered article according to claim 1, wherein the absorbent substrate is a cotton, cotton linter material, cellulose acetate fibers, polyester, polyolefin, and combinations thereof.

14. The multilayered article according to claim 1, wherein the porous substrate is substantially free of an immobilized protein.

15. The multilayered article according to claim 1, wherein the absorbent substrate is fixedly attached to the porous substrate.

16.-34. (canceled)

35. A method for detecting the presence or amount of an analyte in a sample, the method comprising:
(a) combining the sample, a reagent matrix, and a carrier solution in a vessel to form a test sample, the reagent matrix comprising:
(1) a plurality of first capture component, wherein the first capture component comprises a first analyte capture site and a porous substrate binding site; and
(2) a plurality of a second capture component, wherein the second capture component comprises a second analyte capture site; wherein at least one of the first or second capture components comprises a detection medium; and
(3) optionally, a wetting or lysing agent;
(b) contacting the test sample to a porous substrate layer, wherein the porous substrate layer comprises a plurality of grafted groups to the porous substrate layer, wherein the plurality of grafted groups are derived from (i) an acidic monomer comprising at least one acidic group or salts thereof and a monovalent ethylenically unsaturated group selected from a (meth)acrylate or a (meth)acrylamide; (ii) a neutral hydrophilic monomer comprising at least one —O—CH₂CH₂— group and a monovalent ethylenically unsaturated group selected from a (meth)acrylate or a (meth)acrylamide; or (iii) combinations thereof; and
(iii) detecting a signal produced by an aggregation the analyte with the reagent matrix on a surface of the porous substrate layer.

36. The method of claim 35, further comprising a washing step after contacting the test sample to the porous substrate layer.

37. The method of claim 36, wherein the detecting is by visual inspection or by an instrument.

38. The method of claim 35, wherein the method further comprises a positive control.

39. The method of claim 35, wherein the reagent matrix is substantially free of water.

40. (canceled)
